(19) United States
(12) Patent Application Publication
(10) Pub. No.: US 2011/0237930 A1
(43) Pub. Date: Sep. 29, 2011

(54) MRI compatible motor and positioning system

(76) Inventors: Sean Donaldson, Toronto (CA); Cameron Mahon, Georgetown (CA)

(21) Appl. No.: 13/065,106

(22) Filed: Mar. 14, 2011

Related U.S. Application Data
(60) Provisional application No. 61/313,792, filed on Mar. 14, 2010.

(51) Int. Cl. A61B 5/055

(52) U.S. Cl. 600/411

(57) ABSTRACT

A thermal therapy system is provided. The system includes an applicator and a device for supporting and moving the applicator within a patient's body during therapy. Some embodiments include an elongated ultrasound energy applicator that is positioned using a plurality of positioners along a corresponding plurality of axes. Also, a rotational driver or motor is provided to rotate the thermal therapy applicator in the patient's body during treatment. The apparatus is constructed of magnetically-compatible materials and has a design to avoid or minimize interference between the drive components of the apparatus and a MRI imaging system in use during the treatment.

### TECHNICAL FIELD

The present application relates to ultrasound therapy systems, and particularly to the mechanical and electro-mechanical systems, e.g., bearings, motors and actuators for positioning and rotation of said therapy systems before, during and after treatment of a patient.

### BACKGROUND

Ultrasonic transducers have been employed in ultrasound therapy systems to achieve therapeutic heating of diseased and other tissues. Arrays of ultrasound transducers operating to form a beam of ultrasonic energy cause a conversion of sound to thermal energy in the affected tissue areas or treatment volumes, and a subsequent beneficial rise in the temperature in the treatment volumes. With proper monitoring of the heating effect, ultrasound therapy systems can be used to treat harmful cells and to controllably destroy cancerous tumors.

As known to those skilled in the art, ultrasonic transducers are constructed and operated to take electrical power and produce ultrasound energy waves from a surface of a transducer element in a process generally referred to as transduction. The nature and extent of the transduction depends on the material used to construct the transducers, transducer geometry, and the electrical input to the transducers. A common material used in construction of ultrasound transducers is piezo-electric transducer crystal material (lead zirconate titanate, PZT), which comes in several forms.

One challenge in constructing clinically usable systems for image-guided therapy is in constructing the electrical, mechanical, and electro-mechanical support and driving systems for moving and rotating the components of the system.

It is therefore useful to have a motorized apparatus that is compatible with image-guided therapy environments and which can be used to drive an ultrasound therapy applicator for use in such environments.

### SUMMARY

Embodiments hereof are directed to systems and methods for providing a thermal therapy applicator and supporting components that is suitable for ultrasonic thermal therapy treatments in an image-guided environment such as in magnetic resonance imaging (MRI) environments.

Aspects of the present disclosure provide motors, bearings, and other electrical, mechanical and electro-mechanical support systems for driving a thermal therapy applicator.

Some embodiments are directed to magnetic resonance imaging (MRI) compatible apparatus for moving a thermal therapy applicator within a patient, comprising a mechanical platform that supports said thermal therapy applicator; a plurality of translators for translation of said thermal therapy applicator along a corresponding plurality of axes so as to position an ultrasound energy source of said thermal therapy applicator proximal to a diseased tissue region within said patient; and a motor that provides a rotational drive capability to rotate said thermal therapy applicator about an axis of said applicator so as to orient said ultrasound energy source of said thermal therapy applicator about said axis of said applicator, said motor comprising materials adapted for use in or proximal to a magnetic environment of a magnetic resonance imaging (MRI) system.

Other embodiments are directed to thermal therapy system for applying a thermal therapy to a patient, comprising an ultrasonic energy source disposed proximal to a first end of an elongated thermal therapy applicator; a base portion of said thermal therapy applicator proximal to a second end of said thermal therapy applicator; a cradle that supports said base portion, said cradle allowing for rotation of said thermal therapy applicator about an axis of said elongated thermal therapy applicator so that said ultrasonic energy source can be directed towards a programmed direction about said axis; and a motor comprising magnetically-compatible materials for rotating said thermal therapy applicator about said axis substantially without interference with operation of a magnetic resonance imaging (MRI) system in use on said patient.

### BRIEF DESCRIPTION OF THE DRAWINGS

For a fuller understanding of the nature and advantages of the present invention, reference is be made to the following detailed description of preferred embodiments and in connection with the accompanying drawings, in which:

FIG. 1 illustrates an exemplary system for providing image-guided ultrasound therapy to a patient;

FIGS. 2-5 illustrate exemplary views of a MRI-compatible motor apparatus for use in MRI-guided thermal therapy systems or other situations;

FIG. 6 illustrates an exemplary MRI-compatible positioning apparatus including MRI-compatible motor for moving a thermal therapy device; and

FIG. 7 illustrates an exemplary mounting structure for holding and rotating a thermal therapy device in a MRI-compatible positioning apparatus.

### DETAILED DESCRIPTION

As discussed in related applications by the present inventors and assignee, an ultrasound thermal therapy device may be externally controlled so that a therapeutic ultrasound array may be rotated about an axis within a diseased region of a patient. The rotation about an axis may be substantially along an axis of an anatomical passageway such as the male urethra in the example of treating a diseased prostate.

Motorized and computer-controlled movement, positioning and/or rotation of the treatment applicator within the patient may be desired. In the case where the treatment is monitored using magnetic resonance imaging (MRI), the apparatus for positioning and rotating the treatment applicator should be immune to the magnetic field effects of the MRI system, and the treatment applicator controls should not substantially interfere with the MRI images.

Motors relying on magnetic force to provide prime movement are not ideal for use near the bore of an MRI system due to the extreme magnetic fields of the system. Alternative approaches include pneumatic motors, hydraulic motors and piezo-electric motors. The embodiments presented below are directed primarily to a piezo-electric motor and system, but may be in some aspect generalized to other designs.

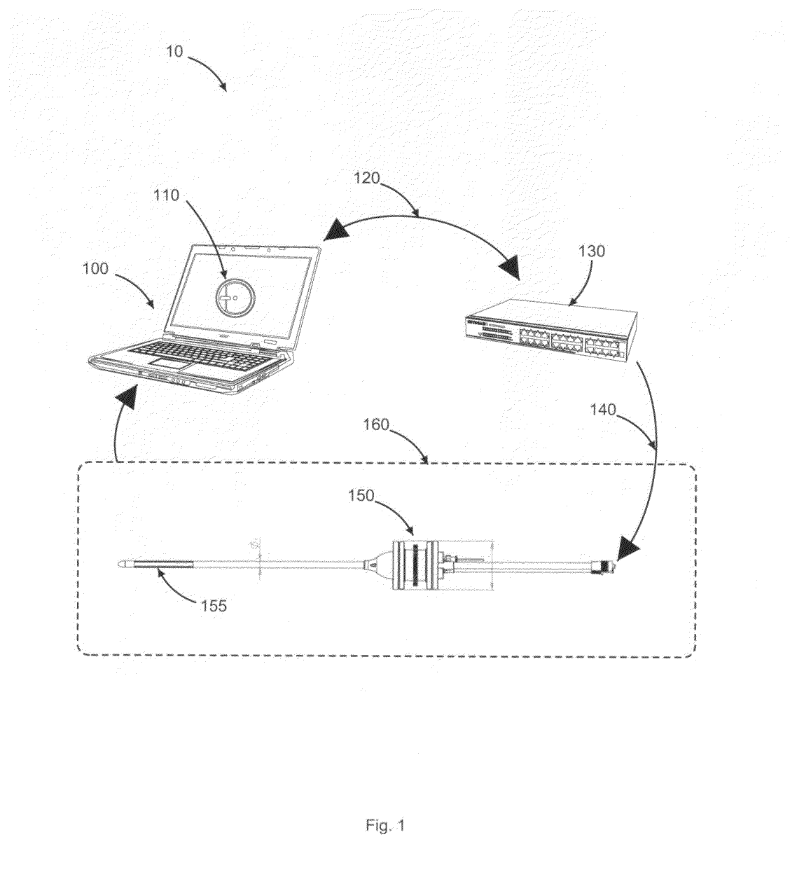

By way of overview of the general clinical application, FIG. 1 illustrates an exemplary system 10 for providing image-guided ultrasound therapy to a patient. The simplified illustration shows a master computer 100, such as a portable PC, workstation, or other processing device having a processor, memory, and coupled to some input/output apparatus. Master computer 100 may include a display and may support a user interface 110 to facilitate control of and observation of the thermal therapy treatment process.

Master computer 100 is adapted for coupling to other systems and components through a computer interface connector 120. Connection 120 carries data and information to and from master computer 100 and may comprise standard or special-purpose electrical wiring connection cables, such as serial connection cables or the like. Also, connection 120 may be achieved wirelessly as known to those skilled in the art of wireless communication, and may further be achieved by way of multiple connections, over a network, or by another suitable method.

In some embodiments, master computer 100 is coupled through connection 120 to a power control unit 130. Power control unit 130 may be implemented as a stand-alone hardware apparatus but may be implemented as a part of master computer 100, e.g., by being built onto a special card in a computer or server system that accommodates such hardware components.

Power control unit 130 may specifically include at least a processor adapted for processing machine or program instructions, which may be provided to the processor from another component of system 10 and may be stored on a memory device in power control unit 130. Circuitry including analog and/or digital circuitry may be operated within power control unit 130 so as to determine an output power to one or more ultrasound therapy transducer elements in an ultrasound therapy apparatus 150.

In some embodiments, power control unit 130 may deliver controlled electrical driving signals to a plurality of ultrasound transducer elements (e.g., PZT array elements) in ultrasound therapy apparatus 150. The driving signals may be controlled to deliver a programmed amount of power to each element or to groups of elements of therapy apparatus 150. The driving signals may also be controlled so as to provide a determined driving voltage, current, amplitude, waveform, or frequency to said ultrasonic transducers of therapy apparatus 150. Such electrical driving signals are carried from power control unit 130 to the ultrasound therapy apparatus 150 over suitable wires, cables, or buses 140. Appropriate plug interfaces or connectors may be included so as to mate the various ends of the connectors or buses to and from their associated components.

In operation, ultrasound therapy apparatus 150 includes a portion 155 that is inserted into a portion of a patient's body to deliver a suitable dose of ultrasound energy to tissue in a diseased region of the patient's body.

The patient and the ultrasound therapy apparatus 150 are generally disposed in an imaging volume 160 such as a magnetic resonance imaging (MRI) apparatus, which can provide real-time images of the relevant parts of the patient, e.g., the treatment volume to master computer 100 or display and user interface 110. In some embodiments, real-time monitoring of the thermal therapy is performed so that a clinical operator can monitor the progress of the therapy within the treatment volume or diseased tissue. Manual or automated changes can be made to the power signals from power control unit 130 based on input from the results and progress of the treatment.

The feedback and coupling of the treatment system components to the control components in system 10 can be used to ensure that an optimum radio frequency (RF) power signal is provided to each element of an ultrasound array 155 used in treatment of diseased tissues. Some examples include treatment of prostate cancer tumors in male patients using MRI guided ultrasound therapy applications.

RF power control unit 130 may include separate circuit cards having individual processors, amplifiers, filters and other components to achieve the desired driving power output to the elements of ultrasound array 155 of ultrasound treatment apparatus 150. Alternatively, a single processor may be employed to control the behavior of the various power channels to each array element.

In the embodiments described below, a fluid system is used to control the temperature of the thermal therapy apparatus or applicator and/or to control a temperature of a region of tissue proximal to the therapy applicator.

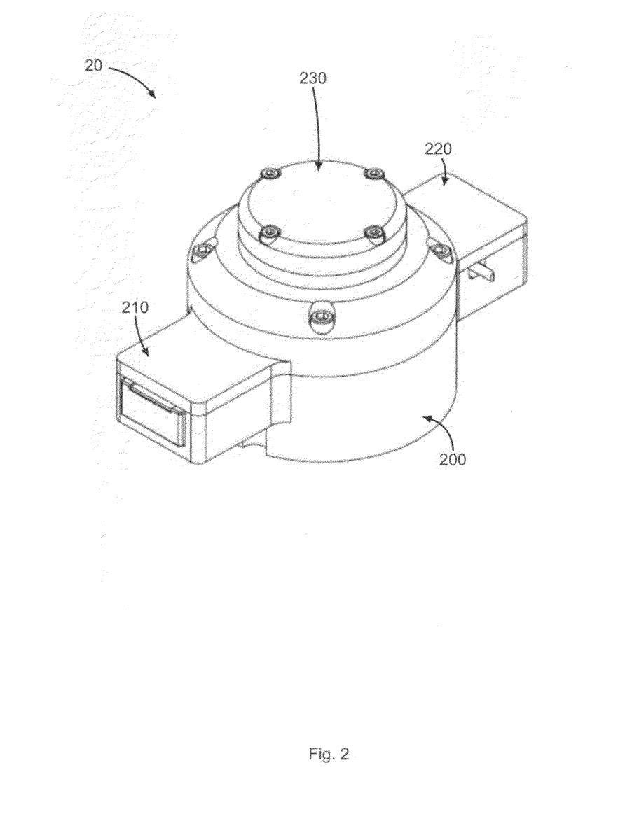

FIG. 2 illustrates a system 20 for providing and controlling fluid delivery to and from a thermal therapy applicator. The fluid may be used to cool (or heat) various components of the thermal therapy applicator. The fluid may also cause the tissues adjacent to the applicator apparatus to be affected so as to prevent unwanted overheating of a region of healthy tissue directly proximal to an ultrasound applicator.

An exemplary MRI-compatible motor 20 is illustrated in perspective in FIG. 2. Motor 20 includes a housing 200 and a pair of opposing piezo drives 210 and 220, which provide prime movement force to cause a desired rotation within motor 20. Piezo drives 210 and 220 may be of a type having small actuating ‘feet’ that make repetitive creeping motions so as to cooperatively move a central shaft (not shown in this view) beneath cap 230 about an axis of motor 20. To encourage and enhance the rotational movement of the motor, a smooth, or engineered surface is provided to an annular ring member within the motor (to be described in more detail below) that permits the slights repetitive motion of the ‘feet’ attached to piezo drivers 210 and 220 to cause a rotation of said annular ring member. In combination, piezo drivers 210 and 220 cause a clockwise (or counter-clockwise) rotation of the annular ring member and coupled central shaft to turn the motor in a given direction about its axis.

In some embodiments, the two opposing piezo drives 210 and 220 are electrically powered so that they operate in phase with one another. For example, the two piezo drives may be wired in parallel to a driving electrical power source.

The design of motor system 20 is such that the two opposing piezo drivers 210 and 220 are in balance, balancing one another, and allowing movement of the motor with minimal friction or resistance. The system may be tuned before use so that the piezo drivers are optimally located and disposed. Furthermore, in some embodiments, the balancing and setup of the piezo drivers is done independently from one another and/or independently from setup of the position sensing encoder, described below. The balancing may be accomplished while the motor is turning, fixing one driver then adjusting the other.

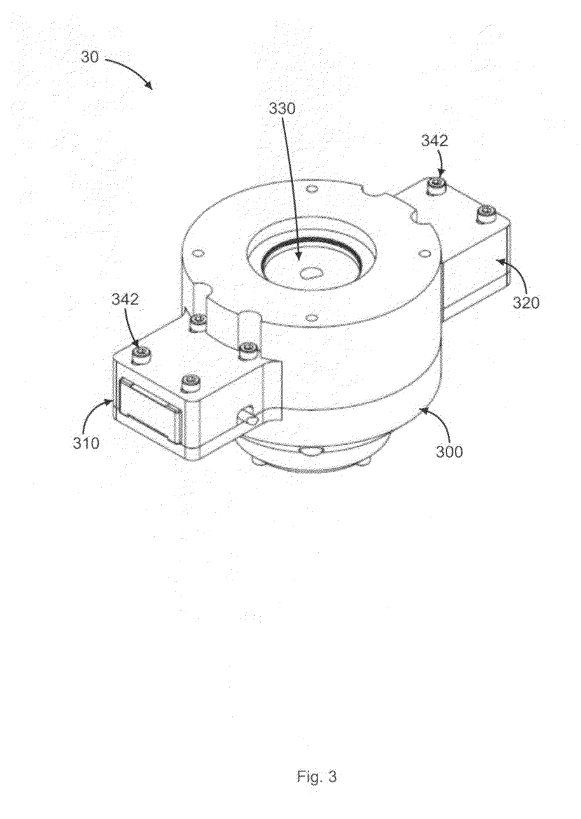

FIG. 3 illustrates another view of MRI-compatible motor system 30. The system includes a housing 300, which may be constructed of a non-ferrous material, a polymer or plastic material, a ceramic material, or other suitable material. First and second piezo drivers 310 and 320 provide prime mover force to rotate central shaft 330 about an axis thereof. Screws 342 hold the parts of the system together and may be substituted by suitable welds, snap joints, or other fasteners or couplings.

It should be noted that using two (or more) opposing drivers (e.g., 310, 320) to rotate motor system 30 allows for a balanced and even and smooth delivery of power. In some embodiments this avoids unwanted displacement or asymmetrical delivery of torque to shaft 330 as might occur if only one driver was used. In some aspects, the use of a pair of opposing piezo electric, or similar, drivers of the present preferred embodiments allows the motor bearings to be thrust roller bearings rather than a conventional bearing that uses highly precise machined components that prevent side-to-side movement.

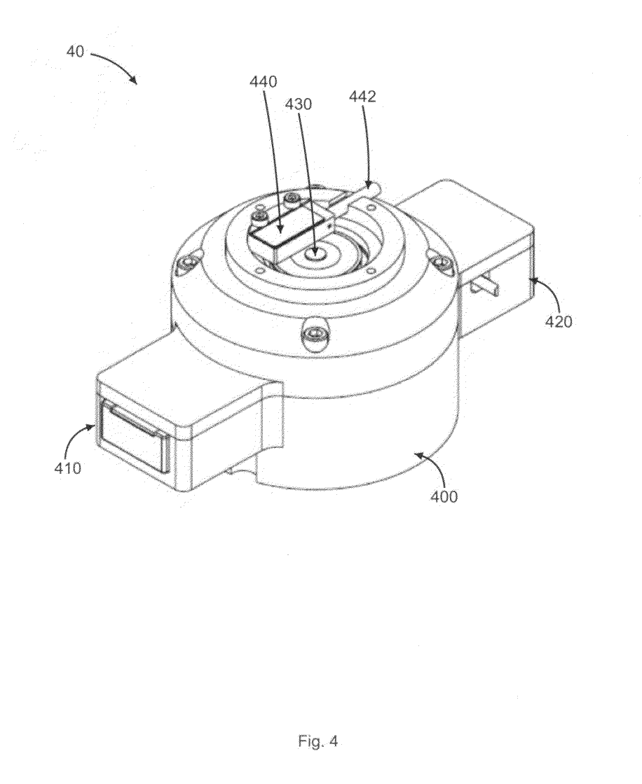

FIG. 4 illustrates a perspective view of a MRI-compatible motor system 40 having a housing 400 supporting two piezo drivers 410 and 420 similar to that described above. The system includes a position sensing encoder 440 that detects the position, or the movement, of the motor shaft as it rotates about a central axis 430. The position sensing encoder 440 is coupled to a conducting signal line 442 to pass information about the position and/or speed of the motor to a control system (not shown).

In some embodiments, the position encoder 440 comprises a grating disk and a pick-up head. The separation between these two components may be set to a suitable distance, e.g., 10-15 thousandths of an inch. The distance between these two components can be tuned using shims substantially without changing any aspect of the inner central shaft or the bearings.

The position encoder 440 may be designed such that the signals are substantially or purely sinusoidal in some embodiments. Sinusoidal signals may not interfere with the MRI system operation as much as other signals. This is consistent with the normal position encoding done in an MRI system.

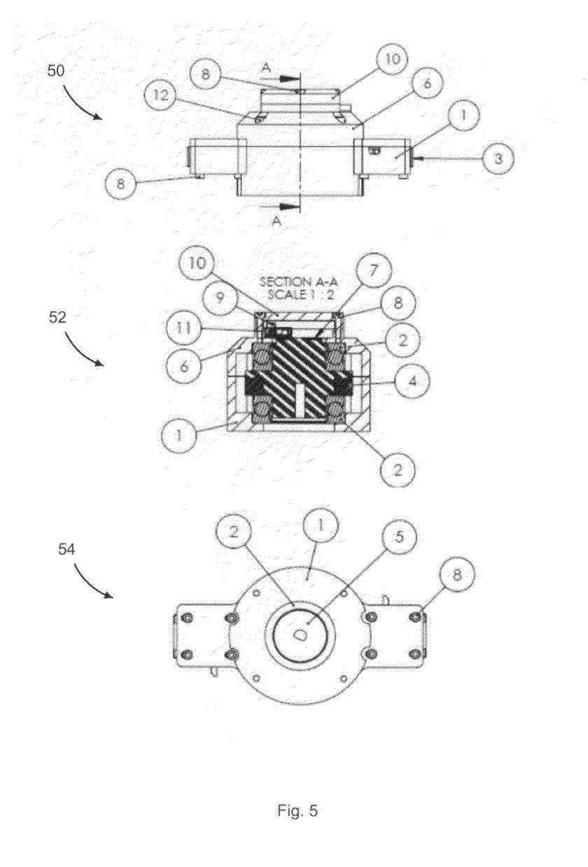

FIG. 5 illustrates three views 50, 52, 54 of a MRI-compatible motor system, where the first view 50 is a side view, the second view 52 is a cross-sectional view of view 50, and the third view 54 is an end-view of the motor system.

Some elements of the motor system that can be seen in FIG. 5 include: motor housing (1), two plastic and glass thrust ball bearings (2), opposing piezo drivers (3), precise annular ring member (4), rotating shaft (5), enclosures (6), (10) secured to housing (1), grating disk (7), and position sensing encoder (11).

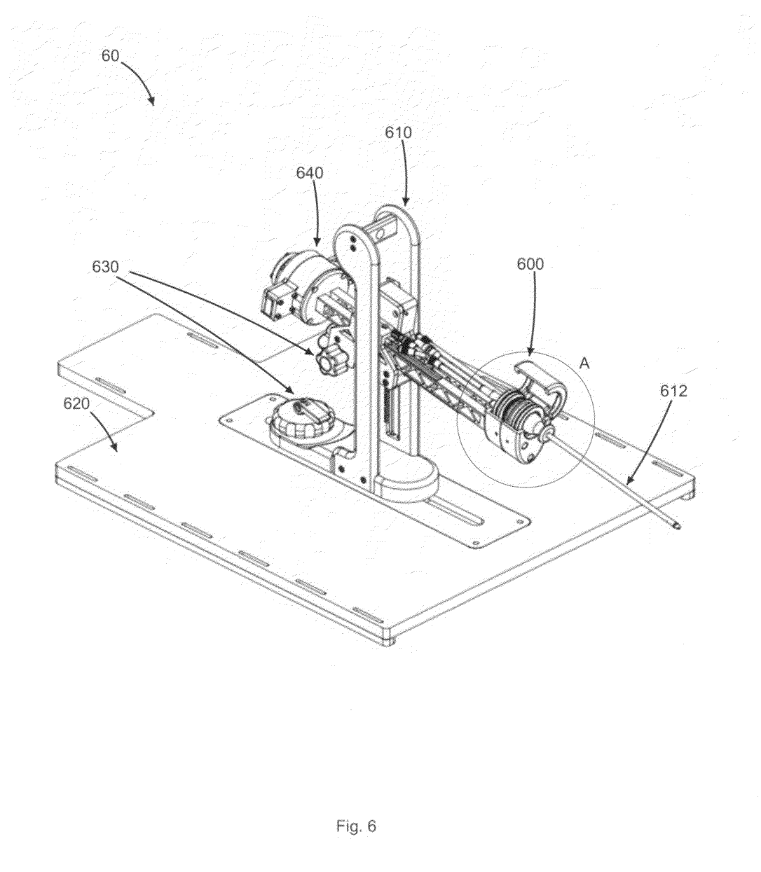

For a better understanding of how the present MRI-compatible motor system is used in a therapeutic system, FIG. 6 presents an exemplary therapy system 60 for treating a patient in a MRI environment.

A thermal therapy applicator includes a handle portion 600 and an elongated portion 612, which is inserted into a patient to be proximal to and/or within a volume of diseased tissue such as the male prostate. Region “A” of the thermal therapy applicator will be illustrated below.

A mechanical positioning apparatus 610 assists clinical operators in positioning the treatment applicator so it can be inserted into a patient's body. The mechanical positioning apparatus 610 is supported on a rigid support plate or platform 620 that in turn may be fixed to a treatment gurney, platform, or table near the patient. Positioning knobs 630 allow for the needed degrees of freedom to position and set the angle of the treatment applicator. For example, the applicator may be moved towards or away from the patient, and may be moved up and down to a determined height, etc. The positioners 630 may be adapted to be electro-mechanical and machine driven if needed, or may be manually set by an operator.

The MRI-compatible motor system 640 can be seen at one end of the therapy system 60, and is used to provide rotational force to turn a shaft that transmits torque down to a set of bearings and gears of therapy applicator 600, as will be seen in detail “A” on the following figure. This allows for cradling of the elongated applicator and for controlled rotation of elongated portion 612 within the volume to be treated so that the treatment can treat a volume surrounding the distal end of elongated portion 612 to the right of FIG. 6. In some embodiments, the elongated portion 612 has near its distal end an ultrasonic or other heating device (array of PZT transducers) that provide heating to treat diseased tissue surrounding the applicator 612.

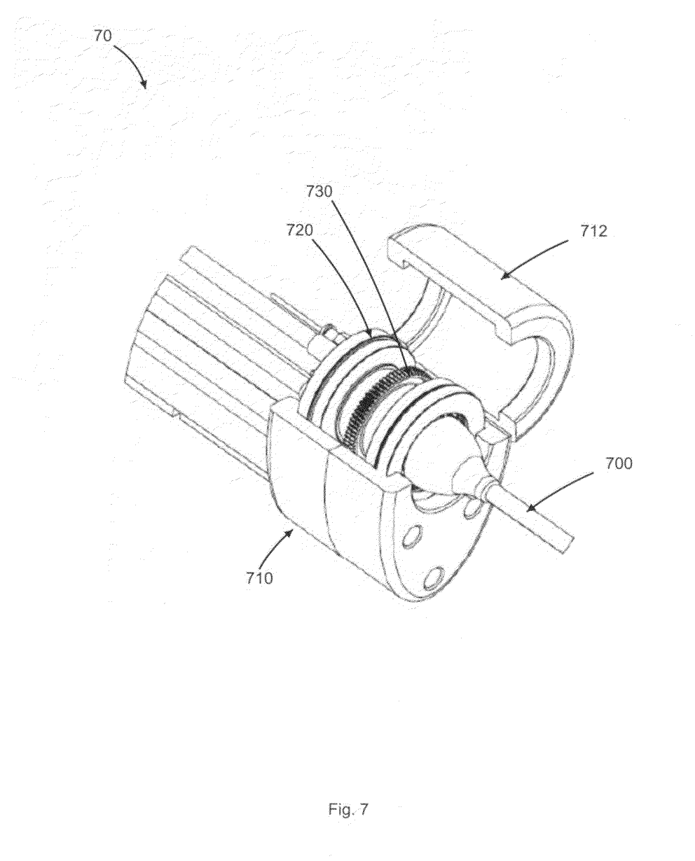

FIG. 7 illustrates a portion 70 corresponding to detail “A” (600) of FIG. 6. MRI-compatible bearings 720 and gears 730 receive torque from the MRI-compatible motor shaft transmitted to portion 70. A lower housing 710 and upper housing 712 may be hinged or fastened so as to be opened for insertion and maintenance of the treatment applicator. Gears 730 couple to gears from the motor shaft to rotate the elongated member 700 of the treatment applicator about its axis, which may be substantially parallel to the rotating axis of the motor.

It is useful to be able to attach and disconnect fluid and electrical connections to the therapy applicator apparatus. For example, temperature control circulation fluids can be provided to and from the apparatus as described in other applications by the present applicant. Also, electrical power and control connections are used in driving the ultrasound applicator array and monitoring certain parameters. Therefore, in some aspects, it is provided by the present embodiments to allow connection and use of electrical and fluid circuits in the present system substantially while using the system, or during and/or before and/or after a treatment. The apparatus may be translated and it may be rotated as necessary to accomplish the treatment within a patient without needing to disconnect or untangle wires and tubing connected to the apparatus. One benefit of this is that the fluid within the fluid tubing can remain sterile and not broken or exposed to the open environment once in use.

The present discussion should not preclude other equivalent or reasonably similar embodiments, and those readers skilled in the art would appreciate that the present embodiments may be generalized to include other types of drivers and components that may be substituted or integrated into or with the present system. The electrical power to drive the prime movers may be provided by a computer controlled circuit and may be programmably adapted to accommodate a therapeutic procedure, including by real-time control of the movement of the motor system. In some embodiments, the movement may be varied as a function of time to change the motor's speed or direction of rotation, and may allow for stopping of the rotation as well when needed.

The present invention should not be considered limited to the particular embodiments described above. Various modifications, equivalent processes, as well as numerous structures to which the present invention may be applicable, will be readily apparent to those skilled in the art to which the present invention is directed upon review of the present disclosure.

We claim:

1. A magnetic resonance imaging (MRI) compatible apparatus for moving a thermal therapy applicator within a patient, comprising:
a mechanical platform that supports said thermal therapy applicator;
a plurality of translators for translation of said thermal therapy applicator along a corresponding plurality of axes so as to position an ultrasound energy source of said thermal therapy applicator proximal to a diseased tissue region within said patient; and
a motor that provides a rotational drive capability to rotate said thermal therapy applicator about an axis of said applicator so as to orient said ultrasound energy source of said thermal therapy applicator about said axis of said applicator, said motor comprising materials adapted for use in or proximal to a magnetic environment of a magnetic resonance imaging (MRI) system.

2. The apparatus of claim 1, said mechanical platform being mechanically fixed with respect to a platform on which said patient is disposed.

3. The apparatus of claim 1, said mechanical platform being mechanically fixed with respect to said magnetic resonance imaging system.

4. The apparatus of claim 1, said motor comprising at least a ceramic portion that avoids or minimizes interaction with a magnetic field of said magnetic resonance imaging system.

5. The apparatus of claim 1, said motor comprising a plurality of piezo electric drivers, each of said piezo electric drivers configured to drive said motor along a respective direction.

6. The apparatus of claim 1, said motor coupled to a thrust roller bearing to allow rotational movement of said applicator and motor about said axis of said applicator.

7. The apparatus of claim 1, further comprising a geared collar that engages a corresponding gear so as to allow rotational movement of said thermal therapy apparatus within said collar during a thermal therapy procedure and that permits coupling of said thermal therapy applicator to a plurality of connections.

8. The apparatus of claim 7, said plurality of connections comprising fluid connections for a fluid passing into and out of said thermal therapy applicator.

9. The apparatus of claim 7, said plurality of connections comprising electrical connections for power and control signals passing into and out of said thermal therapy applicator.

10. The apparatus of claim 7, said plurality of connections comprising both electrical and fluid circuit connections connecting corresponding electrical and fluid circuits within the thermal therapy applicator to respective electrical and fluid portions of said circuits outside of said thermal therapy applicator.

11. The apparatus of claim 1, further comprising an electronic control circuit that controls movement of said translators and motor.

12. The apparatus of claim 1, further comprising a servo device for control and sensing of movement by any of said translators or motor.

13. The apparatus of claim 1, further comprising a cradle portion that cradles and supports a base of an elongated thermal therapy applicator.

14. The apparatus of claim 1, said translators comprising electro-mechanical motorized translators that move the thermal therapy applicator in respective translational degrees of freedom.

15. The apparatus of claim 1, said translators comprising manually-operated positioners that allow moving the thermal therapy applicator in respective translational degrees of freedom.

16. A thermal therapy system for applying a thermal therapy to a patient, comprising:
an ultrasonic energy source disposed proximal to a first end of an elongated thermal therapy applicator;
a base portion of said thermal therapy applicator proximal to a second end of said thermal therapy applicator;
a cradle that supports said base portion, said cradle allowing for rotation of said thermal therapy applicator about an axis of said elongated thermal therapy applicator so that said ultrasonic energy source can be directed towards a programmed direction about said axis; and
a motor comprising magnetically-compatible materials for rotating said thermal therapy applicator about said axis substantially without interference between said motor and a magnetic resonance imaging (MRI) system in use on said patient.
